(19) United States
(12) Patent Application Publication
(10) Pub. No.: US 2024/0090937 A1
(43) Pub. Date: Mar. 21, 2024

(54) ELECTROSURGICAL DEVICE WITH A DEPLOYABLE ELECTRODE TIP AND INTEGRATED MODULATED SMOKE EVACUATION AND SUCTION CONTROL

(71) Applicant: Mohile Innovative Solutions LLC, West Bloomfield, MI (US)

(72) Inventors: Neil Vivek Mohile, West Bloomfield, MI (US); Vivek Vasudev Mohile, West Bloomfield, MI (US)

(73) Assignee: Mohile Innovative Solutions LLC, West Bloomfield, MI (US)

(21) Appl. No.: 18/185,892

(22) Filed: Mar. 17, 2023

Related U.S. Application Data
(60) Provisional application No. 63/408,504, filed on Sep. 21, 2022.

(51) Int. Cl. A61B 18/14

(52) U.S. Cl. CPC A61B 18/1402; A61B 2018/00601; A61B 2218/008

(57) ABSTRACT

An electrosurgical device that comprises a deployable electrode tip and concurrent smoke evacuation and suction control integrated in a single apparatus. An electrode tip is supported for motion so that when a selected button is pressed, the tip extends past a suction tube and is activated for cutting and/or coagulating tissue. In this configuration, the suction tube is in a useful position to evacuate surgical site smoke. When pressure on the selected button is released, the tip moves into a retracted position and is deactivated. This allows for the suction tube to evacuate any remaining smoke and suction fluid/solid debris at the surgical site. Optional suction tube sizes and configurations with a control valve for finger-modulated suction can be exchangeable as per surgical demands. This device would allow surgeons to smoothly transition from surgical dissection with smoke evacuation to fluid and debris suctioning to optimize the surgical process.

### CROSS-REFERENCE TO RELATED APPLICATIONS

This application claims the benefit of and priority to U.S. Provisional Application No. 63/408,504, filed Sep. 21, 2022, entitled Electrosurgical Device with a Deployable Electrode Tip and Integrated Modulated Smoke Evacuation and Suction Control, the contents of which are hereby incorporated by reference as if fully set forth herein.

### BACKGROUND

Electrosurgical devices are widely employed by surgeons in various specialties for surgical dissection and hemostasis. This is accomplished via an electrode tip that transmits a high-frequency electrical current at the surgical site in specific waveforms for either cutting or coagulating tissue. In monopolar systems, a generator delivers electrical energy to an electrosurgical device, then to a grounding pad positioned on the patient's body and subsequently back to the generator to complete the electrical circuit.

A consequence of the use of electrosurgical devices is the generation of smoke at the surgical site. Surgical site smoke not only can obstruct visualization of the surgical field but also can lead to direct inhalation by operating room personnel. The inhalation of surgical smoke poses many hazardous effects including asthma, emphysema, bronchitis, irritation to eyes, nose and throat, headache, allergies, carcinoma of the respiratory tract and others. Consequently, occupational safety and health institutes have advised for the use of smoke evacuation systems in the operating room.

Current electrocautery devices have implemented integrated smoke evacuation tubes or cones in proximity to the electrode tip. However, compliance to these and other smoke evacuation systems remains low. This is due to the fact that they play no role in improving the overall efficiency during surgery. While these devices are sufficient at evacuating smoke during tissue dissection, they are ineffective at suctioning fluid and solid debris, which is imperative during surgery. Thus, independent suction devices are still utilized in addition to these electrosurgical devices, which often crowds the operative field and necessitates multiple surgical assistants and/or repeated instrument exchanges, which is inefficient and costly. Furthermore, as surgeons are aptly focused on the surgery itself, due to the crowding of multiple instruments, suction devices routinely fall from the operative field, causing delays and even compromising the sterile environment.

Current products in the market lack a single device that smoothly transitions from dissecting tissue to suctioning fluid and solid debris, while maintaining a safe smoke-free operating room environment. This invention describes a novel electrosurgical device that allows for operators to dissect tissue while concurrently evacuating smoke and suction fluid/debris with excellent proficiency to ultimately optimize the field of surgery.

### SUMMARY OF THE INVENTION

This invention pertains to a novel electrosurgical device that provides a deployable electrode tip and concurrent smoke evacuation and suction control integrated in a single apparatus. An electrode tip is placed within the electrosurgical device housing and cover assembly, which is supported for motion to be deployed or retracted. The electrosurgical device is connected to a generator which delivers an electrical current in waveforms for cutting and/or coagulating tissue. The housing and cover assembly also contains a channel for removable and interchangeable suction tubes of different sizes and configurations as per surgical demands. The suction tube is connected to a vacuum source which exerts a suction force, which enables smoke evacuation and removal of fluid/solid debris. A window within the cover provides access to a control valve to allow for finger-modulated suction in certain tube configurations. A suction tube without a control valve provides for continuous, non-modulated suction if desired.

When the actionable button is pressed (e.g., “CUT” or “COAG”), the electrode tip is deployed and activated for cutting or coagulating tissue. In this configuration, the electrode tip is extended past the suction tube to allow for effective tissue dissection while ensuring that the suction tube is unobstructive and clear from the field of view during surgery. The suction tube is in a position useful to evacuate surgical site smoke in this deployed state. When pressure on the actionable button is released, the electrode tip moves into a retracted position and is deactivated. In this retracted state, the suction tube can effectively evacuate any remaining smoke along with suction fluid and solid debris at the surgical site. A preferred method to use the described electrosurgical device is by having the thumb control the cutting and/or coagulating function while the index finger is resting on the control valve of the suction tube. This method allows for the operator to surgically dissect tissue while simultaneously modulating suction force for smoke evacuation and fluid/solid debris removal.

A locking mechanism within the housing can be regulated to maintain the electrode tip in the extended locked position. In this configuration, the electrode tip remains extended but is only activated when the actionable button is pressed and deactivated when it is released. The fixed, extended position maintains the electrosurgical device in a state primed for cutting and/or coagulating tissue while evacuating smoke. Additionally, this configuration allows for placement and removal of the electrode tip in a safe, efficient manner while the device is in an extended but not activated position.

This invention allows for operators to smoothly transition from dissecting tissue while evacuating smoke to suctioning fluid and solid debris in a single device. It should be noted that the concepts described may be arranged to form any suitable configuration to produce a functioning electrosurgical device with smoke evacuation/suction. For instance, if desired, the suction tube may be removed to allow for independent operations of both components: the electrosurgical feature (in the deployed/retracted positions or in the fixed extended position) and the suction feature for smoke evacuation and fluid/solid debris removal. Further aspects of this invention are explained via the detailed drawings in conjunction with their associated descriptions which highlights the novel principles of the invention.

### BRIEF DESCRIPTION OF THE DRAWINGS

The invention will be made apparent by the following descriptions in conjunction with the accompanying drawings, in which:

FIG. 1A shows an isometric view of the assembled Electrosurgical Device in a retracted state. FIG. 1B shows an isometric view of the assembled Electrosurgical Device in an extended state with a magnified view of the Window Opening and Vacuum Control Valve.

FIG. 2 is an exploded view with subassemblies of the Electrosurgical Device.

FIG. 3A and FIG. 3B show isometric views of Suction Tubes with and without a Vacuum Control Valve, respectively.

FIG. 4 shows a cross-section of the Suction Tube in the Electrosurgical Device assembly.

FIG. 5A, FIG. 5B and FIG. 5C show the locking mechanism for the Suction Tube with arrows indicating the action required to lock the Suction Tube into the Housing & Cover Subassembly.

FIG. 6A shows a cross-section of Electrode Tip, Cone Tip, Carrier Subassembly, Printed Circuit Board (PCB) Subassembly and Solenoid Subassembly in a retracted state.

FIG. 6B shows a cross-section of Electrode Tip, Cone Tip, Carrier Subassembly, PCB Sub Assembly and Solenoid Subassembly in an extended state.

FIG. 6C shows a radial cross-section which shows Housing, Cover Subassembly, Carrier Subassembly, PCB Sub Assembly and Electrical Spring Contacts.

FIG. 7A shows an isometric view of the Electrode Tip in a retracted position and FIG. 7B shows an isometric view of the Electrode Tip in an extended position.

FIG. 8A and FIG. 8B show cross-sections of the connection between the Carrier Subassembly, Solenoid Subassembly, Cover Sub Assembly and Housing in retracted and extended positions, respectively.

FIG. 9A and FIG. 9B show cross-sections of the Electrosurgical Device indicating the mechanical Lock Button in the unlocked and locked positions, respectively.

FIG. 10 shows a cross-section of the Electrosurgical Device indicating the Cut and Coagulate buttons, PCB assembly, Electrical Spring Contacts within the Carrier Subassembly and the Electrode Tip.

FIG. 11 shows an alternative configuration of the Electrosurgical Device with a Pneumatic Valve to actuate the Electrode Tip from a retracted to extended position instead of the Solenoid Subassembly.

FIG. 12 shows an alternative configuration of the Electrosurgical Device with an Electric Motor to actuate the Electrode Tip from a retracted to extended position instead of the Solenoid Subassembly.

### DETAILED DESCRIPTION

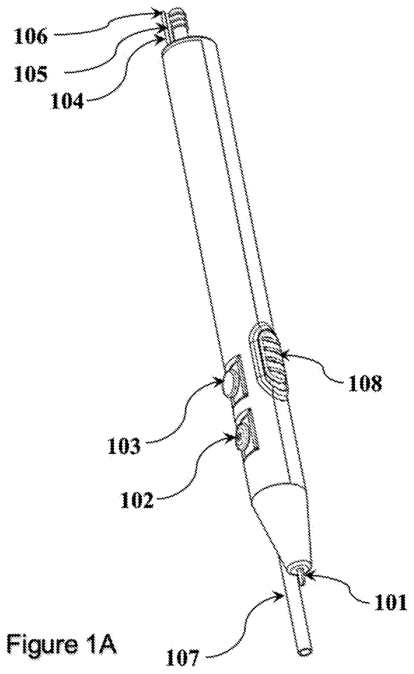

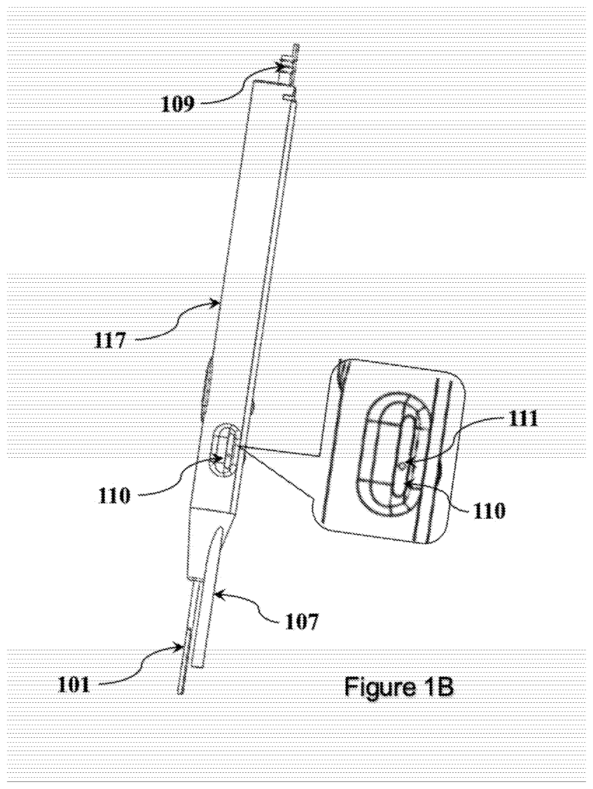

The present invention is directed to this Electrosurgical Device and more specifically, an integration of multiple functions including cutting/coagulating tissue, smoke evacuation, fluid suction and vacuum level control. This single device allows for multiple configurations, which are required for specific surgical situations and needs. The Electrosurgical Device is shown in FIG. 1A and FIG. 1B.

FIG. 1A shows the assembled Electrosurgical Device with the Electrode Tip 101 in the retracted position. When either the Cut Button 102 or Coagulate Button 103 is pressed, the Electrode Tip 101 extends past the Suction Tube 107 into the extended position, as shown in FIG. 1B, and thereby activates the Electrode Tip 101. When the Electrode Tip 101 is activated, electrical energy is provided to the Electrode Tip 101 through an external generator via separate Electrical Wire Conductors 104, 105, 106 based on which button (Cut Button 102 or Coagulate Button 103) is pressed. Holding either the Cut Button 102 or Coagulate Button 103 maintains the Electrode Tip 101 in the extended and activated position. Releasing either the Cut Button 102 or Coagulate Button 103 moves the Electrode Tip 101 back to the retracted position, thereby deactivating the Electrode Tip 101. The Electrode Tip 101 can only be activated in the extended position when either the Cut Button 102 or Coagulate Button 103 is pressed. When the Lock Button 108 is mechanically actuated, the Electrode Tip 101 is locked in an extended position. In this configuration, the Electrode Tip 101 is activated by pressing either the Cut Button 102 or Coagulate Button 103 but remains in the extended position. When the Lock Button 108 is unlocked, the Electrode Tip 101 moves back to the retracted position.

FIG. 1B shows an isometric view of the Electrosurgical Device with the Electrode Tip 101 in the extended position. There is a Suction Tube 107 with a Vacuum Tube Interface 109 which is connected to an external vacuum source. This Vacuum Tube Interface 109 has multiple serrations for proper sealing of the external vacuum source. The Vacuum Tube Interface 109 is integrated into the Suction Tube 107 as a one-piece design. The magnified view shows a Window Opening 110 within the Cover Subassembly 117. The Vacuum Control Valve 111 within the Suction Tube 107 is exposed through the Window Opening 110. The vacuum level in the Suction Tube 107 can be modulated by partially or fully covering the Vacuum Control Valve 111 with a finger during surgery.

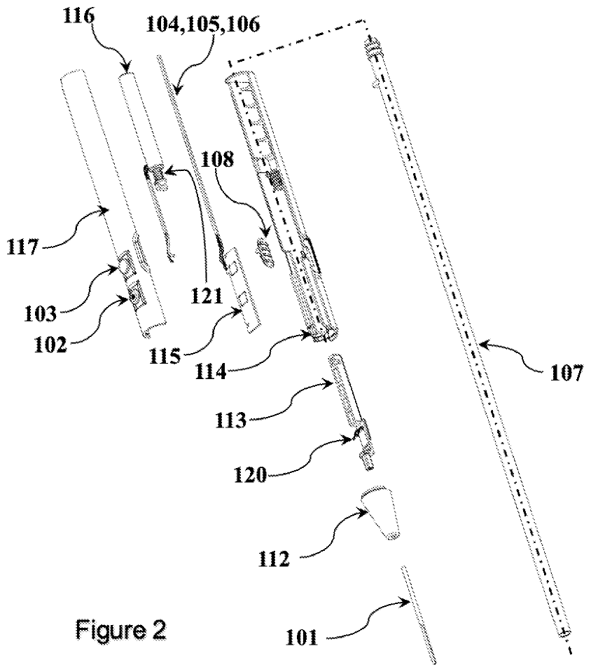

FIG. 2 is an exploded view of the complete assembly and highlights all components, features and sub-assemblies of the Electrosurgical Device. The Electrode Tip 101 is inserted into the Carrier Sub Assembly 113 through the opening within the Cone Tip 112. The Carrier Sub Assembly 113 slides within the Housing 114 and Cover Sub Assembly 117 to move the Electrode Tip 101 between retracted and extended positions. The Carrier Subassembly 113 also provides an interface for the Lock Button 108 to lock the Carrier Subassembly 113 and Electrode Tip 101 in the extended position. In the extended position, the Carrier Sub Assembly 113, via integrated Electrical Spring Contacts 120, makes electrical contact with the PCB Sub Assembly 115. The Carrier Subassembly 113 is mechanically connected to the Solenoid Subassembly 116. The Solenoid Subassembly 116, via the Solenoid Spring 121, maintains the Carrier Subassembly 113 and Surgical Tip 101 in a retracted position in the deactivated state, unless the Lock Button 108 is actuated. When the Cut Button 102 or Coagulate Button 103 (which is part of the Cover Assembly 117) is pressed, an electrical contact is made on the PCB Sub Assembly 115, which sends an electrical signal to the Solenoid Subassembly 116 to activate and move the Carrier Subassembly 113 and Electrode Tip 101 to the extended position. The PCB Subassembly 115 provides regulated electrical signals to activate the Solenoid Subassembly 116. The PCB Subassembly 115 provides an electrical contact and energy through the Electrical Spring Contacts 120 which are within the Carrier Subassembly 113 and then to the Electrode Tip 101 for cutting or coagulating tissue. The PCB Sub Assembly 115 is connected via Electrical Wire Conductors 104, 105 and 106 to an external generator which provides electrical energy. The Suction Tube 107 is inserted into the Electrosurgical Device Housing 114 and Cover Sub Assembly 117 and securely locked with a cam. Multiple configurations are available for the Suction Tube 107, including different sizes, shapes and with or without the Vacuum Control Valve 111 (shown in FIG. 1B). The combined Housing 114 and Cover Sub Assembly 117 provides a channel to insert the Suction Tube 107 and lock it in place. The cam allows for an easy, quick exchange of different Suction Tubes 107 based on surgical needs.

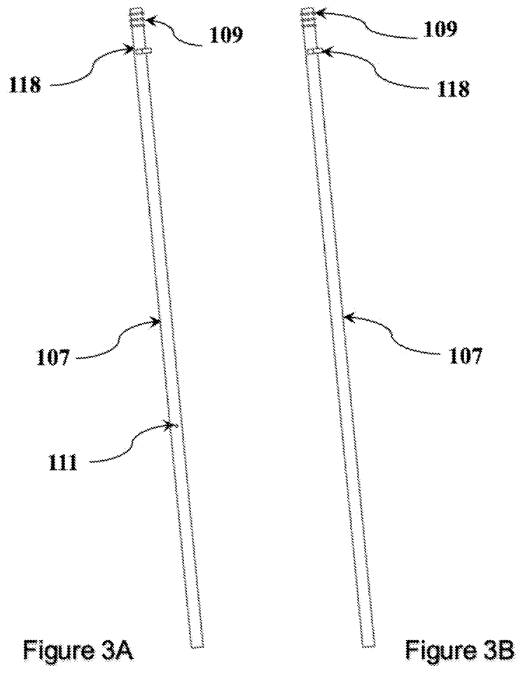

FIG. 3A shows an isometric view of the Suction Tube 107. The Suction Tube 107 is inserted into the Electrosurgical Device Housing 114 and the Cover Assembly 117 and through the Cone Tip 112 (as shown in FIGS. 2 and 1B). The Cam 118 is integrated into the Suction Tube 107. The Suction Tube 107 has a Vacuum Control Valve 111 to modulate the vacuum level by partially or covering it with a finger during surgery. Multiple configurations of the Suction Tube 107, including different sizes or shapes, are available and able to be inserted within the same channel of the Electrosurgical Device. The Vacuum Tube Interface 109 is integrated into the Suction Tube 107 and is securely sealed to an external vacuum.

FIG. 3B shows an isometric view of an alternative configuration of the Suction Tube 107 without a Vacuum Control Valve 111 (as shown in FIG. 3A) if modulation of the vacuum level is not required. This configuration has the integrated Cam 118 and Vacuum Tube Interface 109.

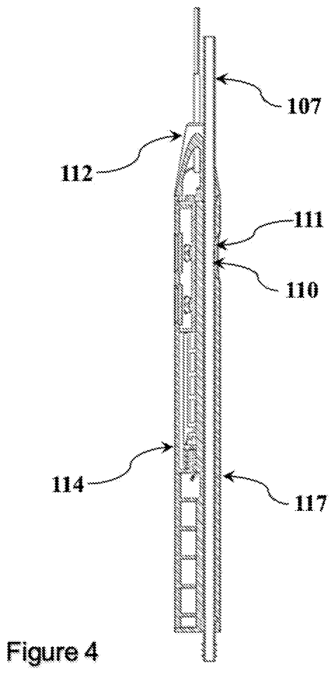

FIG. 4 shows a longitudinal cross-section of the Suction Tube 107 inserted into the channel created by Housing 114 and Cover Sub Assembly 117 and through the Cone Tip 112. The Vacuum Control Valve 111 is exposed through the Window Opening 110 of the Cover Assembly 117 for finger-modulated vacuum control.

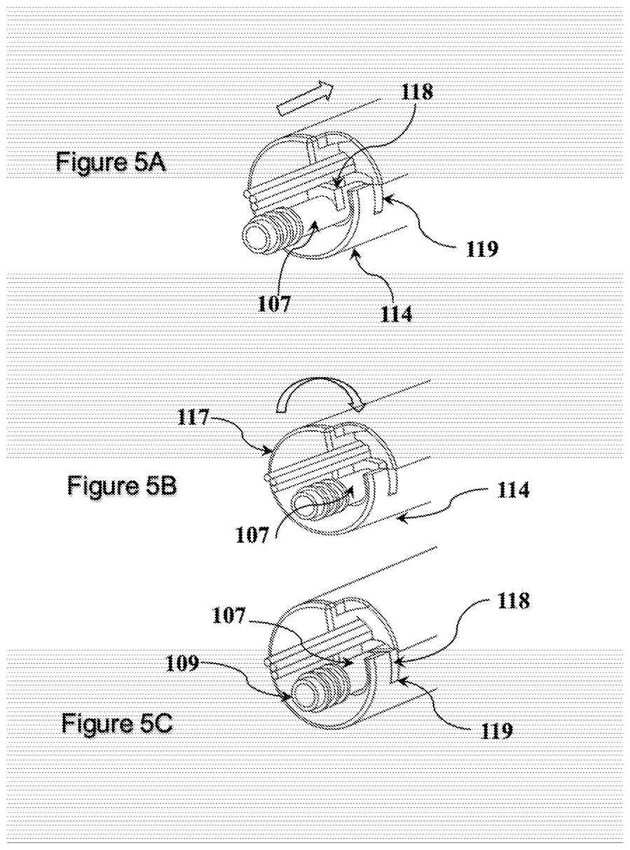

FIGS. 5A, 5B and 5C show the locking mechanism and mechanical action required to lock the Suction Tube 107 into the Housing 114. In FIG. 5A, the Suction Tube 107 is longitudinally inserted (see arrow) within the channel of the Electrosurgical Device Housing 114 and Cover Sub Assembly 117 (shown in FIG. 5B). The Cam 118 is not engaged with the Housing Slot 119. In FIG. 5B, the Suction Tube is rotated (see arrow) to engage the Cam 118 (shown in FIG. 5A) with the Housing Slot 119 (shown in FIG. 5A), thereby locking it into position. FIG. 5C shows the Cam 118 and Housing Slot 119 fully engaged and securely locked into the Electrosurgical Device assembly. In this locked state, the Vacuum Control Valve 111 is exposed within the Window Opening 110 (shown in FIG. 1B). The external vacuum source is connected to the Vacuum Tube Interface 109.

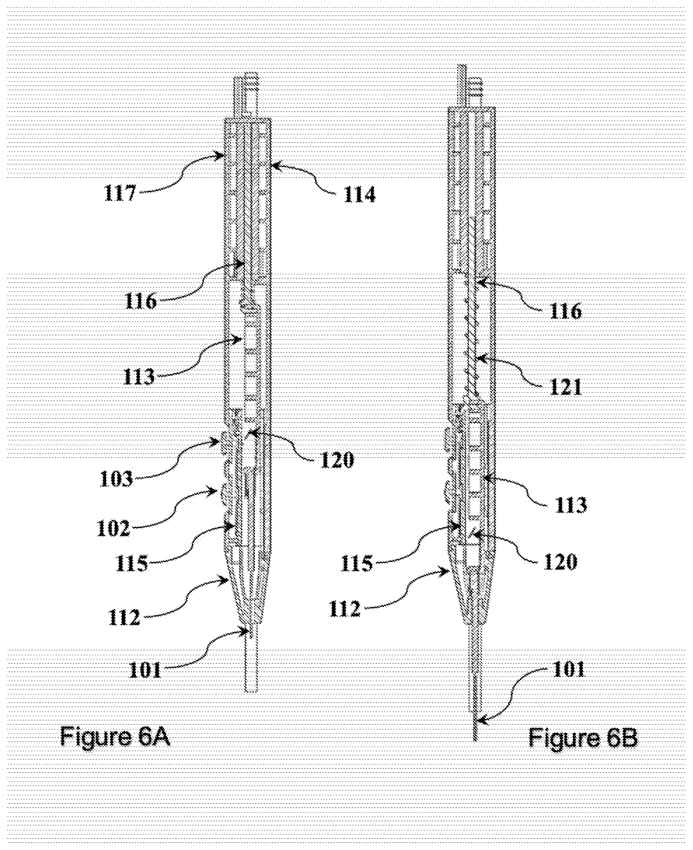

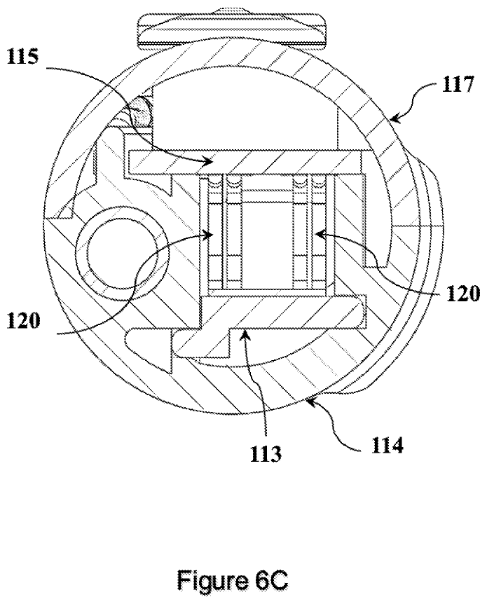

FIG. 6A shows a cross-section of Electrode Tip 101, Cone Tip 112, Carrier Sub Assembly 113, PCB Sub Assembly 115 and Solenoid Subassembly 116 in a retracted state. These components are held in place by the Housing 114 and Cover Sub Assembly 117. The removable Electrode Tip 101 is inserted through the Cone Tip 112 and into the Carrier Subassembly 113 and makes an electrical contact with the Carrier Subassembly 113. When either the Cut Button 102 or Coagulate Button 103 button is pressed, the PCB Subassembly 115 provides regulated electrical energy to activate the Solenoid Subassembly 116, which then drives the Carrier Subassembly 113 from the retracted to extended position. In the extended position, the Electrical Spring Contacts 120 of the Carrier Subassembly 113 make an electrical contact with the PCB Sub Assembly 115, which provides electrical energy to the Electrode Tip 101 to cut or coagulate tissue. The Carrier Sub Assembly 113 slides from the retracted to extended position within the Housing 114 and Cover Sub Assembly 117.

FIG. 6B shows a cross-section of Electrode Tip 101, Cone Tip 112, Carrier Sub Assembly 113, PCB Sub Assembly 115 and Solenoid Subassembly 116 in an extended state. Holding either the cut or coagulate button or locking the Carrier Subassembly 113 will maintain the Electrode Tip 101 in the extended position. The Electrical Spring Contacts 120 of the Carrier Sub Assembly 113 are only in contact with the PCB Sub Assembly 115 in the extended state and can therefore provide energy to the Electrode Tip 101. When the Solenoid Subassembly 116 is activated, electrical energy will overcome the force of the Solenoid Spring 121 to move the Carrier Subassembly 113 and Electrode Tip 101 to the extended position. When the Solenoid Subassembly 116 is deactivated (by releasing the cut or coagulate button), the Solenoid Spring 121 brings the Carrier Subassembly 113 and Electrode Tip 101 to the retracted position (unless locked by the Lock Button 108 shown in FIG. 1A).

FIG. 6C shows a radial cross-section of the Housing 114, Cover Sub Assembly 117, Carrier Sub Assembly 113, PCB Sub Assembly 115 and Electrical Spring Contacts 120. The Carrier Subassembly 113 is held by the Cover Subassembly 117 and the Housing 114 so that it can move in a longitudinal direction within the Electrosurgical Device from a retracted to extended position. FIG. 6C shows the Electrical Spring Contacts 120 of the Carrier Sub Assembly 113 making an electrical contact with the PCB Sub Assembly 115, which when activated by pressing the Cut Button 102 or Coagulate Button 103 (not shown), provides electrical energy to the Electrode Tip 101 (not shown) to cut or coagulate tissue.

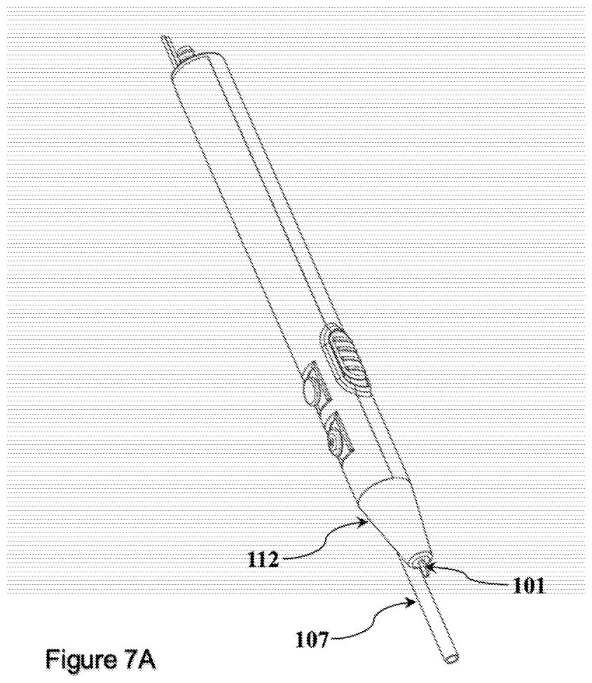

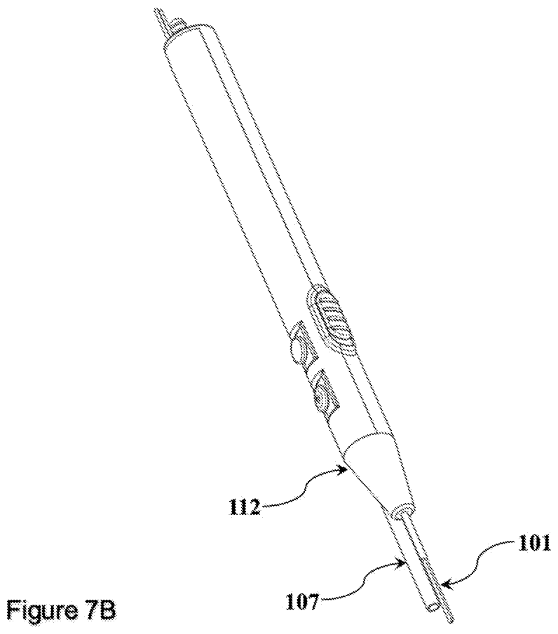

FIG. 7A shows the Electrode Tip 101 in the retracted position, Cone Tip 112 and Suction Tube 107. In this configuration, the suction tube is effective at suctioning fluid and solid debris at the surgical site. The vacuum suction level can be modulated via the Vacuum Control Valve 111 (not shown). Alternative Suction Tube 107 configurations of different sizes or shapes can be selected as per surgical needs. The Electrode Tip 101 is in the retracted position and is therefore unobstructive to allow for appropriate suctioning of the surgical site.

FIG. 7B shows the Electrode Tip 101 in the extended position (past the Suction Tube 107) and the Cone Tip 112. In this configuration, the Electrode Tip 101 can cut or coagulate tissue based on pressing the respective button. The Suction Tube 107 is able to evacuate smoke generated at the surgical site. Since the Electrode Tip 101 extends past the Suction Tube 107, the Suction Tube 107 is unobstructive to allow for appropriate tissue dissection.

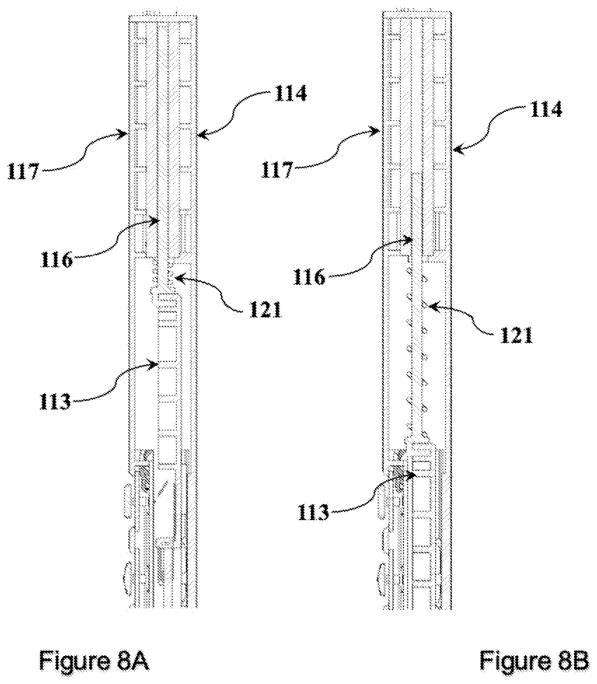

FIG. 8A shows a cross-section of the connection between the Carrier Sub Assembly 113, Solenoid Subassembly 116, Cover Sub Assembly 117 and Housing 114 in the retracted position. When the Solenoid Subassembly 116 is in the deactivated state, the Solenoid Spring 121 maintains the Carrier Subassembly 113 and Electrode Tip 101 (not shown) in the retracted position. This position is the resting state for the Solenoid Subassembly 116. The Carrier Subassembly 113 is mechanically connected to the Solenoid Assembly 116 and slides between the retracted and extended position within the assembly guided by the Housing 114 and Cover Sub Assembly 117.

FIG. 8B shows a cross-section of the connection between Carrier Sub Assembly 113, Solenoid Assembly 116, Cover Sub Assembly 117 and Housing 114 in extended position. When the Solenoid Subassembly 116 is in activated position, the Solenoid Spring 121 is stretched and slides the Carrier Subassembly 113 and Electrode Tip 101 (not shown) into the extended position.

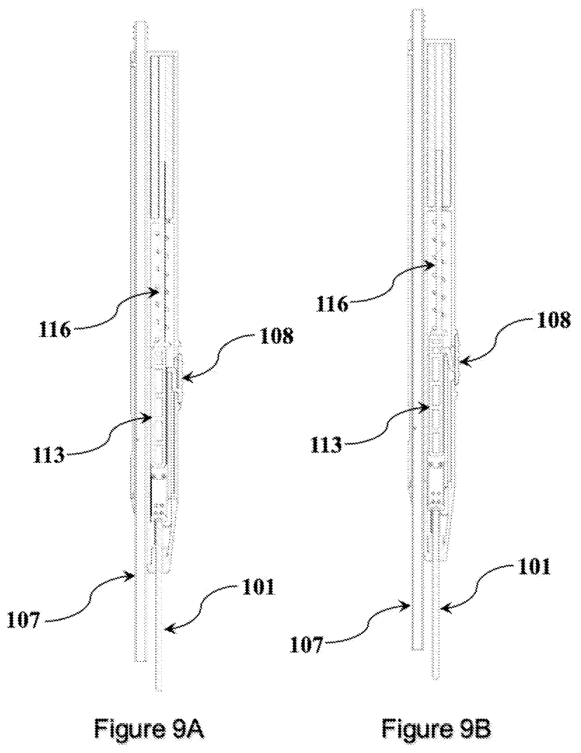

FIG. 9A shows the cross-section of the Electrosurgical Device indicating the Lock Button 108 in the unlocked position. The Electrode Tip 101 is in the activated and extended position since either the cut or coagulate button is pressed and held, thereby activating the Solenoid Subassembly 116. When the Lock Button 108 is unlocked, the Carrier Subassembly 113 and the Electrode Tip 101 is able to move between the retracted and extended positions based on the respective deactivated or activated state of the Solenoid Subassembly 116. The smoke evacuation via the Suction Tube 107 remains independently active via the external vacuum source.

FIG. 9B shows a cross-section of the Electrosurgical Device indicating the Lock Button 108 in the locked position. When the Lock Button 108 is locked, the Carrier Subassembly 113 and the Electrode Tip 101 are fixed in the extended position, regardless of whether the Solenoid Subassembly 116 is in the activated or deactivated state. This configuration allows the Electrode Tip 101 to remain in the extended position even when the cut or coagulate button is not pressed. The Electrode Tip 101 will only be activated to cut or coagulate tissue when the respective button is pressed. The Lock Button 108 can be unlocked at any time to allow the Carrier Sub Assembly 113 and Electrode Tip 101 to move to the retracted position. The smoke evacuation via the Suction Tube 107 remains independently active via the external vacuum source.

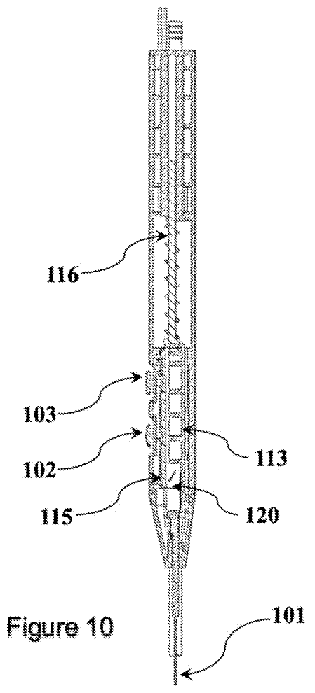

FIG. 10 shows a cross-section of the Electrosurgical Device indicating the Cut Button 102 and Coagulate Button 103. The Carrier Subassembly 113 and Electrode Tip 101 are in the extended position. When the Cut Button 102 or Coagulate Button 103 is pressed, the Solenoid Subassembly 116 is activated to move the Carrier Subassembly 113 and the Electrode Tip 101 to the extended position. In this extended configuration, an electrical contact is made between the Electrical Spring Contacts 120 of the Carrier Subassembly 113 and the PCB Assembly 115. The electrical contact allows the external electrical generator to provide appropriate energy to the Electrode Tip 101 to cut or coagulate tissue.

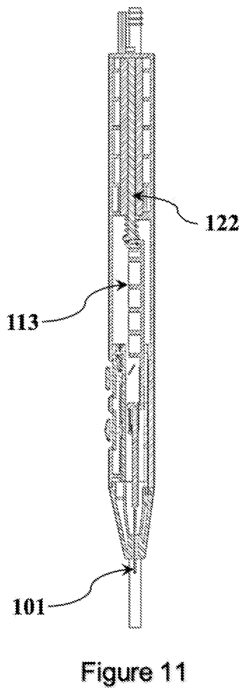

FIG. 11 shows an alternative configuration of the Electrosurgical Device with a Pneumatic Valve 122 instead of a Solenoid Subassembly 116 (not shown) in the retracted position. In this configuration, an external source provides pressurized air to activate the Pneumatic Valve 122 to move the Carrier Subassembly 113 and Electrode Tip 101 to the extended and activated position. With no air pressure, the Pneumatic Valve 122 is deactivated and the Carrier Sub Assembly 113 and Electrode Tip return to the retracted position via a spring. This is an alternative mechanism of the Electrosurgical Device.

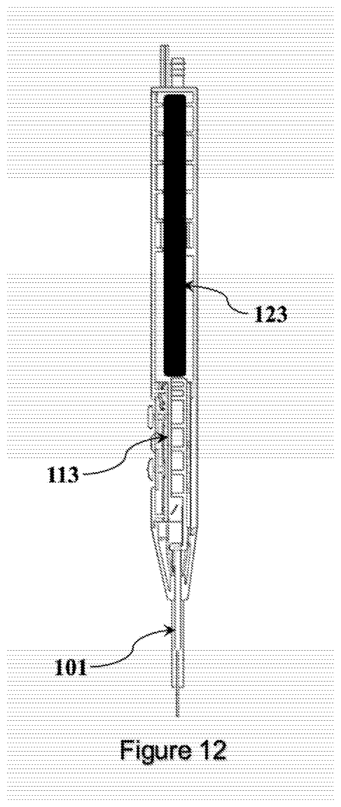

FIG. 12 shows an alternative configuration of the Electrosurgical Device with an Electric Motor (with a position sensor) 123 instead of a Solenoid Subassembly 116 (not shown) in the retracted position. In this configuration, an external source provides electrical energy to activate the Electric Motor 123 to move the Carrier Subassembly 113 and Electrode Tip 101 to the extended and activated position. When either the cut or coagulate button is released, the Electric Motor 123 returns the Carrier Subassembly 113 and Electrode Tip 101 to the retracted position. This is an alternative mechanism of the Electrosurgical Device.

Having described the invention, the following is claimed:

1. An Electrosurgical Device with integrated smoke evacuation and suction, the electrosurgical device comprising:
a) An assembled unit connected to an external generator that produces electrical currents in waveforms for cutting or coagulating tissue activated via two respective buttons;
b) An Electrode Tip placed within the assembled unit, which is supported for motion so that the Electrode Tip moves from a retracted position to an extended position, relative to the assembled unit; and
c) The assembled unit contains a housing unit including a channel for an insertable and interchangeable smoke evacuation Suction Tube with a suction tip, which is fixed and locked into place with a connection to an external vacuum source.

2. The Electrosurgical Device of claim 1, wherein the Suction Tube apparatus comprises various sizes and configurations that are interchangeable within the same Electrosurgical Device.

3. The Electrosurgical Device of claim 2, wherein the Suction Tube contains a cam to insert and lock the tube within the housing unit channel.

4. The Electrosurgical Device of claim 2, wherein the Suction Tube is assembled to connect to different suction tip sizes and configurations.

5. The Electrosurgical Device of claim 2, wherein the Suction Tube contains a valve where the operator can modulate the suction force by manually (partially or fully) covering the valve, thereby controlling vacuum level within the Suction Tube.

6. The Electrosurgical Device of claim 5, wherein the housing unit contains a window that provides access to the Suction Tube control valve for suction force modulation.

7. The Electrosurgical Device of claim 1, wherein the Electrode Tip is insertable and removable within a sliding apparatus of the housing unit.

8. The Electrosurgical Device of claim 7, wherein the sliding apparatus and the Electrode Tip translate in a longitudinal direction, between the retracted and extended positions.

9. The Electrosurgical Device of claim 7, wherein the housing unit includes a Solenoid Subassembly, to move the sliding apparatus and the Electrode Tip between the retracted and extended positions.

10. The Electrosurgical Device of claim 1, wherein in the retracted position, the Electrode Tip remains drawn back relative to the suction tip.

11. The Electrosurgical Device of claim 1, wherein a Solenoid Subassembly within the housing unit is connected to a sliding apparatus which exerts a spring force to maintain the sliding apparatus and inserted Electrode Tip in the retracted position.

12. The Electrosurgical Device of claim 1, wherein in the extended position, the electrode tip deploys longitudinally past the suction tip to perform a cut or coagulate function.

13. The Electrosurgical Device of claim 1, wherein the housing unit contains a Cut Button that when manually depressed, produces an electrical current to generate a force against a spring to move a sliding apparatus and inserted Electrode Tip to the extended position; when the sliding apparatus moves to the extended position, an electrical contact is made to produce an electrical current in specific waveforms, activating the Electrode Tip and enabling it to cut tissue.

14. The Electrosurgical Device of claim 13, wherein the housing unit contains a Coagulate Button that when manually depressed, produces an electrical current to generate a force against a spring of a solenoid assembly to move the sliding apparatus and inserted Electrode Tip to the extended position; when the sliding apparatus moves to the extended position, an electrical contact is made to produce an electrical current in specific waveforms, activating the Electrode Tip and enabling it to coagulate tissue.

15. The Electrosurgical Device of claim 14, wherein when pressure on the Cut Button or Coagulate Button is released, the spring of the Solenoid Subassembly exerts a force to move the sliding apparatus and inserted Electrode Tip into the retracted position; when the sliding apparatus moves to the retracted position, electrical contact is lost and the Electrode Tip is deactivated.

16. The Electrosurgical Device of claim 1, wherein a sliding apparatus is assembled to be mechanically locked in the extended position by activating a lock button.

17. The Electrosurgical Device of claim 16, wherein in the locked, extended position, a Cut Button is manually depressed to produce an electrical current in specific waveforms, activating the Electrode Tip and enabling it to cut tissue.

18. The Electrosurgical Device of claim 17, wherein in the locked, extended position, a Coagulate Button is manually depressed to produce an electrical current in specific waveforms, activating the Electrode Tip and enabling it to coagulate tissue.

19. The Electrosurgical Device of claim 18, wherein in the locked, extended position, when pressure on the Cut Button or the Coagulate Button is released, the electrode tip is deactivated and the sliding apparatus and Electrode Tip remain in the locked, extended position.

20. The Electrosurgical Device of claim 16, wherein in the locked, extended position, the sliding apparatus can be mechanically unlocked, and move from the extended position to the retracted position.

21. The Electrosurgical Device of claim 1, wherein the housing unit includes a piston, moveable by a pneumatic force, to move a sliding apparatus and the electrode tip between the retracted position and the extended position.

22. The Electrosurgical Device of claim 1, wherein the housing unit includes an electric motor, to move a sliding apparatus and the electrode tip between the retracted position and the extended position.

23. A method of utilizing the electrosurgical device of claim 1 includes:
removing the Suction Tube from the housing unit to perform independent operations of the Electrosurgical Device, the independent operations include:
tissue dissection via the Electrode Tip in the extended position; and
smoke evacuation and/or suction via the Suction Tube.

24. A method of providing smoke evacuation as well as suction during an electrosurgical procedure includes using the electrosurgical device according to claim 1 to evacuate, through suction from the external vacuum source, smoke through the suction tube of the electrosurgical device.
